(19) United States
(12) Patent Application Publication
(10) Pub. No.: US 2015/0164943 A1
(43) Pub. Date: Jun. 18, 2015

(54) APPLICATOR DEVICES FOR SKIN CARE PRODUCTS

(71) Applicant: MARY KAY INC., Addison, TX (US)

(72) Inventors: Daniel CHEN, Shanghai (CN); James SWANZY, Arlington, TX (US)

(21) Appl. No.: 14/572,234

(22) Filed: Dec. 16, 2014

Related U.S. Application Data
(60) Provisional application No. 61/917,149, filed on Dec. 17, 2013.

(51) Int. Cl. A61K 33/06; A61F 13/40; A61K 9/00; A61K 31/60; A61K 31/327

(52) U.S. Cl. CPC A61K 33/06; A61K 31/60; A61K 31/327; A61K 9/0014; A61M 35/006

(57) ABSTRACT

Disclosed is a styptic composition formulated for topical application to an acne lesion, the composition comprising a sufficient amount of a styptic to seal the lesion within 30 seconds after topical application, and an anti-acne agent.

### CROSS REFERENCE TO RELATED APPLICATIONS

This application claims the benefit of U.S. Provisional Application 61/917,149, filed Dec. 17, 2013. The entire contents of the referenced application are incorporated into the present application by reference.

### BACKGROUND OF THE INVENTION

A. Field of the Invention

The invention relates generally to styptic compositions that can be used to treat acne lesions. The compositions include an anti-acne agent and a sufficient amount of a styptic to stop bleeding from or to seal such lesions within 30 seconds after contact.

B. Description of Related Art

Acne is commonly referred to as a disorder of the pilosebaceuous unit, which includes the hair follicle, seabaceous gland, and sebaceous duct. Propionibacterium acnes (“P. acnes”), an anaerobic bacteria, is present in all types of human skin and is part of the skin's natural sebum maintenance system. The outward signs of acne on skin has been categorized as noninflammatory and inflammatory acne. Noninflammatory acne typically includes the presence of whiteheads and blackheads (respectively referred to as closed and open comedones). These comedones are compact masses of keratin, sebum, and bacteria, which dilate the follicular duct. Inflammatory acne, by comparison, has been characterized by papules (pimples), pustules, and nodulocystic lesions which can lead to scarring of the skin.

One of the issues with treating inflammatory acne is that the lesions (e.g., pimples, pustules, nodulocystic lesions) tend to be open skin wounds and anti-acne agents tend to be skin irritants. This oftentimes results in a painful burning sensation when applying anti-acne agents to acne lesions. The current solution is to incorporate anti-acne agents into skin soothing vehicles or compositions such as gels, creams, or lotions. Unfortunately, this can dilute the efficacy of the acne agent.

Further, and as noted above, inflammatory acne lesions are oftentimes open skin wounds, in which blood is present and can be expelled from the wound. This can lead to difficulties in applying a typical anti-acne gel, cream, or lotion. In particular, the composition can mix with the blood and result in an unsightly mess on a person's skin. Even further, the flow of the blood out of the wound can cause the anti-acne agent to also exit the wound rather than remain where it is needed.

### SUMMARY OF THE INVENTION

A solution has been discovered to the current drawbacks for treating acne lesions, such as those from inflammatory acne. In particular, the solution is premised on combining an anti-acne agent with a styptic to treat the acne and close or seal the acne lesion at the same time. In particular aspects, the anti-acne agent can be included into known styptic compositions, thereby avoiding the dilutive effect typically seen with acne gels, creams, and lotions. This allows for the direct application of the anti-acne agent to the acne lesion followed by immediate closure of the lesion through the styptic composition. The end result is that the anti-acne agent is trapped within the lesion to provide its healing properties. Further, the bleeding can be stopped in a short period of time (e.g., less than 30 seconds after topical application). All of this can be achieved with a single composition.

In one aspect of the present invention, there is provided a styptic composition formulated for topical application to an acne lesion. The composition includes an anti-acne agent and a sufficient amount of a styptic to close or seal the lesion soon after contact. In particular embodiments, the wound is closed or sealed within 120 second, 60 second, 45, second, 30 second, 15 seconds, 10 seconds, or within 5 seconds after contact. In preferred embodiments, the wound is closed or sealed within 30 seconds after contact. By closure or sealed, it is meant that active bleeding from the wound is stopped or that a seal is formed over the wound. The compositions of the present invention can include any desired amount of the anti-acne agent and a sufficient amount of the styptic to close or seal the wound. By way of example, the compositions can include 50 to 95% by weight of the styptic and 1 to 10% by weight of the anti-acne agent. Such compositions can further include water such as 10 to 50% by weight of water. Alternatively, the compositions can include 10 to 50% by weight of the styptic and 1 to 10% by weight of the anti-acne agent. Such compositions can further include water such as 50 to 90% by weight of water. The ratio of styptic to water can be adjusted to produce flowable liquid formulations, semi-solid formulations, or solid formulations. By way of example, increasing the ratio of styptic to water results in semi-solid to solid formulations. Such formulations typically have a viscosity of 150,000 cps or greater, as measured by Brookfield Viscometer using a TC spindle at 2.5 rpm at 25° C. In more particular aspects, such formulations have a viscosity of 200,000, 250,000, 300,000, 400,000, 500,000, or 1,000,000 cps or greater. By comparison, decreasing the ratio of styptic to water results in more liquid or flowable compositions, which typically have a viscosity of less than 150,000, as measured by Brookfield Viscometer using a TC spindle at 2.5 rpm at 25° C. In more particular aspects, such liquid or flowable compositions have a viscosity of less than 100,000, 90,000, 80,000, 70,000, 60,000, 50,000, 40,000, 30,000, 20,000, 10,000, 5,000, or 1,000 cps or less. In one aspect, the composition is in the form of a solid stick that can be wetted and then applied directed to the acne lesion. In other aspects, the form is of a semi-solid gel or flowable liquid that can be directly applied to said acne lesions. Non-limiting examples of anti-acne agents include antibacterial agents (e.g., azelaic acid, benzoyl peroxide, hydroxyquinoline), keratolytic agents (e.g., glycolic acid, salicylic acid, benzoyl peroxide), antibiotics (e.g., clindamycin, dapsone, erythromycin, sulfacetamide, tetracyclines such as lymecycline, minocycline, doxycycline), retinoids (e.g., adapalene, isotretinoin, motretinide, tazarotene, tretinoin), mesulfen, tioxolone, and any combination thereof. In preferred aspects, the anti-acne agent can be salicylic acid, benzoyl peroxide, benzoic acid, or tretinoin. In even more preferred aspects, the anti-acne agent can be salicylic acid or benzoyl peroxide or a combination of the two. Non-limiting examples of anti-hemorrhaging agents include styptics such as aluminum sulfate, potassium alum, titanium dioxide, or zinc chloride. In preferred aspects, the anti-acne agent is salicylic acid or benzoyl peroxide and the styptic is aluminum sulfate. Excipients can also be added to the compositions of the present invention to help solubilize anti-acne agents that may be poorly soluble in water (e.g., salicylic acid, benzoyl peroxide, tretinoin, etc.). Such excipients include urea and sodium citrate, which can be used alone or in combination with other co-solvents (e.g., glycerin, propylene glycol, PEG 300 and PEG 400). The excipients can be added as needed to obtain the desired level of solubility of the anti-acne agent and the amount of water in the formulation can be adjusted accordingly. Alternatively, the anti-acne agents can simply be suspended within the formulation. The compositions of the present invention can also include additional ingredients that can be beneficial to treating acne. For instance, acne has the potential to leave scars and pigmented skin. Therefore, the compositions of the present invention can include agents that help to prevent scar formation or that help to prevent hyperpigmented or discolored skin. Exam The compositions of the present invention can also include additional ingredients that can further help with treating acne. For instance, the compositions can include skin lightening or brightening agents to aid in the prevention and treatment of dark spots, skin discoloration that can be caused by acne lesions. Non-limiting examples of skin lightening or brightening agents include botanical or plant extracts (e.g., navy bean extract-see U.S. Pat. No. 8,481,090, which is incorporated by reference), hydroquinone and derivatives thereof (e.g., monobenzyl ether of hydroquinone), azelaic acid, kojic acid (5-hydroxy-4-pyran-4-one-2-methyl), mequinol (4-hydroxyanisole), retinoids, niacinamide, soy, vitamin C and derivatives thereof (e.g., magnesium ascorbyl phosphate, ascorbyl-2-glucoside), corticosteroids, licorice and extracts thereof, hydroxystilbene, aloesin, glutathione, glycolic acid, N acetyl glucosamine, gentisic acid, green tea and extracts thereof, melatonin, etc. The compositions can also include anti-irritants or anti-erythemic agents, anti-aging ingredients, or other skin actives.

In another embodiment of the present invention there is disclosed a device that is configured to apply the compositions of the present invention to acne lesions. In some embodiments, the composition is in solid form and is disposed lengthwise within an elongated housing of said device. Alternatively, the composition can be in liquid form and be comprised within a reservoir of said device, wherein the device further includes an absorbent applicator end piece in fluid communication with said reservoir. In either instance, the applicator device can be an elongated device. In instances, where the composition is a solid or semi-solid, the device can be configured to dispose the composition from one end of the device, much like a lipstick or lip gloss applicator works. For instance, the solid or semi-solid composition can be forced out of the elongated housing by twisting the housing or otherwise pushing the composition out of an open end of the applicator device. The composition can then be directly applied to the acne lesion or can be wetted first and then be applied to the acne lesion. In instances where the composition is a liquid, the composition can be loaded onto an absorbent applicator (e.g., a sponge), which can then be used to directly apply the composition to an acne lesion. The device can be configured such that absorbent applicator is in fluid communication with the liquid composition, thereby allowing for continuous absorption of the liquid by the absorbent applicator. Alternatively, a wicking material can be attached to the absorbent applicator and also in contact with the liquid composition. In either instance, the applicator device can be configured to hold or contain the liquid material via an internal storage container or reservoir. In even further embodiments, the applicator device can be a pen, pencil, brush or other material that can be used to absorb the composition onto and then can be used to apply the composition to the acne lesion (e.g., the composition can be a liquid or semi-solid contained within a container and the device can be a brush in which one end includes bristles that can be dipped into the composition, absorb composition, and then the bristles can be applied or brushed onto the acne lesion).

Also disclosed is a method of treating acne lesions or sealing acne lesions, closing acne lesions, reducing or preventing bleeding from an acne-lesions, and the like, by using the compositions of the present invention. The method can include contacting any one of the compositions of the present invention to an acne lesion. Any one of the devices of the present invention can be used in this method. An “open acne lesion” is any pimple, comedone, black head, white head, papule, nodule, pustule, inflammatory lesion, cyst, or nodulocystic lesion wherein the skin has been broken. In preferred aspects, the open acne lesion is an inflamed or inflammatory acne lesion (e.g., pimples, pustules, nodulocystic lesions). In some embodiments, the composition is delivered to the skin in such a way that the majority of it contacts the lesion rather than skin around said lesion. The lesion can be closed and trap the anti-acne agent within said lesion within 5, 4, 3, 2, 1, minute(s), or 45, 30, 15, 10, or 5 second after contact.

Also disclosed in the context of the present invention are embodiments 1 to 20. Embodiment 1 is a styptic composition formulated for topical application to an acne lesion, the composition comprising an anti-acne agent and a sufficient amount of a styptic to seal the lesion within 30 seconds after topical application of the composition to the acne lesion. Embodiment 2 is the styptic composition of embodiment 1, comprising 50 to 95% by weight of the styptic and 1 to 10% by weight of the anti-acne agent. Embodiment 3 is the styptic composition of embodiment 2, further comprising 10 to 50% by weight of water. Embodiment 4 is the styptic composition of embodiment 1, comprising 10 to 50% by weight of the styptic and 1 to 10% by weight of the anti-acne agent. Embodiment 5 is the styptic composition of embodiment 4, further comprising 50 to 90% by weight of water. Embodiment 6 is the styptic composition of any one of embodiments 1 to 5, wherein the composition is formulated as a solid stick. Embodiment 7 is the styptic composition of any one of embodiments 1 to 5, wherein the composition is formulated as a semi-solid. Embodiment 8 is the styptic composition of embodiment 7, wherein the composition has a viscosity of greater than 150,000 cps as measured by Brookfield Viscometer using a TC spindle at 2.5 rpm at 25° C. Embodiment 9 is the styptic composition of any one of embodiments 1 to 5, wherein the composition is formulated as a liquid. Embodiment 10 is the styptic composition of embodiment 9, wherein the composition has a viscosity of less than 150,000 cps as measured by Brookfield Viscometer using a TC spindle at 2.5 rpm at 25° C. Embodiment 11 is the styptic composition of any one of embodiments 1 to 10, wherein the styptic is aluminum sulfate and the anti-acne agent is salicylic acid or benzoyl peroxide. Embodiment 12 is the styptic composition of any one of embodiments 1 to 10, wherein the styptic is aluminum sulfate, potassium alum, titanium dioxide, or zinc chloride, or any combination thereof. Embodiment 13 is the styptic composition of any one of embodiments 1 to 10 or 12, wherein the anti-acne agent is an antibacterial agent (e.g., azelaic acid, benzoyl peroxide, or hydroxyquinoline, or combinations thereof), a keratolytic agent (e.g., glycolic acid, salicylic acid, benzoyl peroxide, or combinations thereof), an antibiotic (e.g., clindamycin, dapsone, erythromycin, sulfacetamide, tetracyclines such as lymecycline, minocycline, doxycycline, or combinations thereof), a retinoid (e.g., adapalene, isotretinoin, motretinide, tazarotene, or tretinoin, or combinations thereof), mesulfen, or tioxolone, or any combination thereof. Embodiment 14 is the styptic composition of any one of embodiments 1 to 14, further comprising a skin lightening ingredient. Embodiment 15 is the styptic composition of any one of embodiments 1 to 14, further comprised in a device configured to apply the composition to the acne lesion. Embodiment 16 is the styptic composition of embodiment 15, wherein the composition is in solid form and is disposed lengthwise within an elongated housing of said device. Embodiment 17 is the styptic composition of embodiment 15, wherein the composition is in liquid form and comprised within a reservoir of the device, wherein the device further comprises an absorbent applicator end piece in fluid communication with said reservoir. Embodiment 18 is a method of sealing or treating an acne lesion on a person's skin comprising contacting the acne lesion with any one of the styptic compositions of embodiments 1 to 17 to said acne lesion, wherein said composition seals the acne lesion within 30 seconds after application. Embodiment 19 is the method of embodiment 18, wherein the acne lesion is bleeding when contacted with the composition and stops bleeding within 30 second after contact. Embodiment 20 is the method of any of embodiments 18 to 19, wherein the composition is applied to the acne lesion via an absorbent applicator end piece of an applicator device.

It is contemplated that any embodiment discussed in this specification can be implemented with respect to any method or composition of the invention, and vice versa. Furthermore, compositions of the invention can be used to achieve methods of the invention.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers. The figures are not drawn to scale.

FIG. 1 is a section taken along the length of an applicator device in accordance with the invention.

FIG. 2 shows a partially cut-away view of the end of an alternative applicator device in accordance with the invention.

FIG. 3 illustrates a brush that can be used to apply a composition of the present invention to an open acne lesion.

### DETAILED DESCRIPTION OF THE INVENTION

The current formulations used to treat acne lesions can dilute the effectiveness of the anti-acne agent. For instance, the structure of the formulation can limit the amount of the anti-acne agent that can be added to the formulation (e.g., a substantial amount of a cream or lotion emulsion is dedicated to obtaining stabilized continuous and discontinuous phases). Further, the current formulations fail to seal the wound in a sufficient amount of time, which can cause the anti-acne agent from flowing out or away from the lesion along with the exiting blood or exudate.

The present invention offers a solution to the current methods and formulations used to treat acne. This solution is based on loading styptic compositions with anti-acne agents. This combination results in a formulation that can be loaded with high amounts of anti-acne agents and that has the ability to quickly seal an open acne lesion, thereby trapping the anti-acne agent within the lesion. This increases the effectiveness of the acne treatment by focusing the active at the site of the lesion while also preventing the unsightly combination of blood with typical anti-acne formulations. Even further, the formulations of the present invention can be loaded into standard styptic pen/pencil devices, which allows for the targeted treatment of acne lesions without having to contact other surfaces of the skin that do not need said treatment. It results in a more efficient use of the compositions of the present invention by limiting treatment to skin surfaces that need it.

These and other non-limiting aspects of the present invention are provided in the following sections.

### A. Applicator Devices

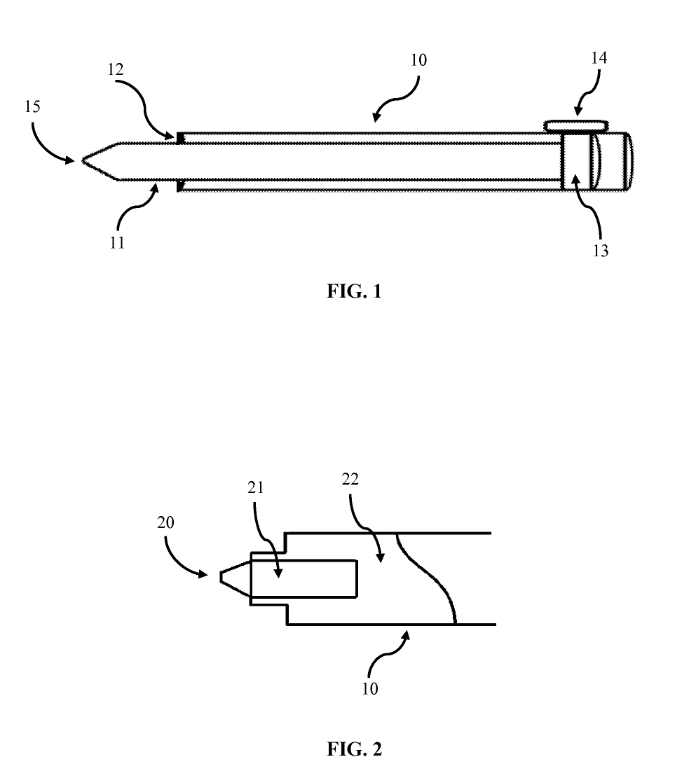

FIG. 1 depicts a cross section view of a non-limiting example of an applicator device according to the present invention. A solid anti-hemorrhagic and anti-acne composition of the present invention is formed into an elongated applicator stick 11. The applicator stick 11 can be, for example, a dry solid formulated to create a paste on its surface when wetted. The paste can then be applied to an open acne lesion by contacting the lesion with the wetted applicator stick 11. The applicator stick can also be a soft solid or semi-solid composition of a consistency similar to lip balm, for example, that can deliver an effective amount of the composition without prior wetting of the stick. Solid or semi-solid applicator sticks 11 can be made by several processes known by persons of ordinary skill in the art. As a non-limiting example, a method of making a solid stick including styptic active ingredients is provided in U.S. Pat. No. 459,738, which is hereby incorporated by reference. Briefly, powdered active ingredients are mixed with gum arabic and water, creating a paste that can be formed into a desired shape. After drying, the composition holds its shape. The applicator stick 11 can be of any suitable shape. The housing 10 can be adapted to accommodate the shape of the applicator stick 11 and vice-versa. The applicator stick 11 can be of any suitable size that allows for application of the active ingredient to a limited area of skin while avoiding application to unaffected areas. To facilitate application to a limited area of skin, the applicator stick 11 may be tapered to a point at the tip 15, as in the embodiment shown. In the embodiment shown, the applicator stick 11 is disposed within an elongated housing 10. The housing 10 may be of any suitably rigid material such that it retains its shape. As non-limiting examples, the housing 11 may be made of plastic, metal, wood, or ceramic. In the embodiment shown, the housing 10 has a collar 12 in contact with the applicator stick 11 such that the collar 12 provides lateral support to the applicator stick 11 to help prevent the applicator stick 11 from moving perpendicularly in relation to the elongated axis of the housing 10 during use. In the embodiment shown, the elongated housing 11 is large enough in relation to the applicator stick 11 that the applicator stick 11 can slide in relation to the housing 10. In some embodiments, the housing can also be in direct contact with the applicator stick such that the applicator stick is in a fixed position relative to the housing. In the embodiment shown, the applicator stick 11 is attached to a base 13 slidably disposed within the housing 10. In the embodiment shown, the base 13 is connected to a slider 14 disposed on the outside surface of the housing 10. By moving the slider 14 in relation to the housing 10, a user can expose more or less of the applicator stick 11 as desired. The slider 14 and base 13 can be attached to one another or can be provided as a single piece.

FIG. 2 depicts a partially cut-away view of the end portion of another non-limiting example of an applicator device in accordance with the present invention. In the embodiment shown, an applicator end piece 20 is disposed at the end of an elongated housing 10. The applicator end piece 20 is in fluid communication via a wick 21 with a reservoir 22 containing a liquid composition including anti-hemorrhagic and anti-acne active ingredients. The applicator end piece 20 is composed of a porous material which enables fluid from the reservoir 22 to flow by capillary action to the surface of the end piece 20, where it is available to be delivered onto the subject's skin. The porous material may be, for example, felt, expanded foam, or POREX. POREX is a moldable porous material produced by or on behalf of Porex Technologies Corporation and comprises a sintered mass of thermoplastic polymer pellets. The wick 21 is also composed of a porous material that enables fluid from the reservoir to flow through it by capillary action. The wick 21 can be the same or different material as the applicator end piece 20. If the wick 21 is the same material, the applicator end piece 20 and the wick 21 can be two pieces joined together, or can instead be a single piece. In some embodiments, the reservoir 22 can contain a free-flowing liquid. The reservoir 22 can also be composed of fibrous fluid retaining material. In such embodiments, the fibers can be manufactured from thermoplastic polymers such as, for example, polyesters, nylons, polypropylenes, and mixtures thereof. The reservoir 22 can be of any dimensions suitable to store a desired amount of liquid composition and fit within the housing 10.

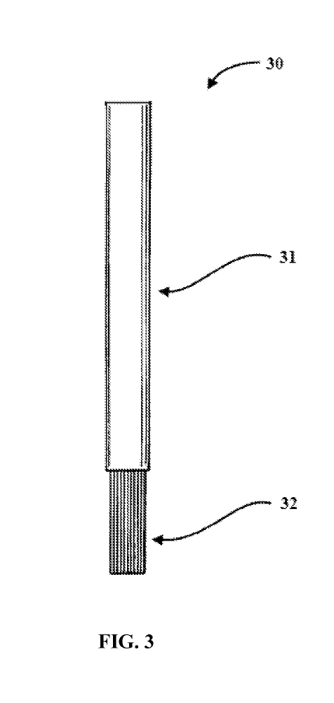

FIG. 3 depicts yet another applicator device of the present invention. In this embodiment, the applicator device 30 is a brush that has an elongated handle 31 and bristles 32 attached to one end of the handle 31. The bristles 32 can be dipped into a composition of the present invention (e.g., the composition can be in liquid form and contained within a standard jar), absorb the composition, and then be used to directly apply the composition to an acne lesion. In this sense, a standard paint brush, eyeliner brush, or mascara brush can be used as the applicator device.

### B. Compositions

The active ingredient compositions of the present invention include both anti-hemorrhagic and anti-acne active ingredients. Examples of anti-hemorrhagic active ingredients include styptics such as aluminum sulfate, potassium alum, titanium dioxide, or zinc chloride. Examples of anti-acne active ingredients include salicylic acid, salicylate salts, benzoyl peroxide, benzoic acid, or tretinoin, including other described throughout the specification. The precise formulations that include these active ingredients depends on the form in which the active ingredients are delivered to the affected area of the skin. For example, the compositions of the present invention can be formulated to be a solid, soft solid, or semi-solid, such as would be used to form the applicator stick 11 in the applicator device illustrated in FIG. 1. The active ingredients can be present in solid formulations in any concentration effective to stop bleeding and promote healing of an acne lesion. Determining such effective concentrations is within the skill of those of ordinary skill in the art. The anti-hemorrhagic active ingredient may be included in amounts of 1, 2, 3, 4, 5, 6, 7, 8, 9, 10, 20, 30, 40, 50, 60, 70, 80, 90, or 95 wt % or any range therein. The anti-acne active ingredient may be included in the formulation in amounts of 0.5, 1, 1.5, 2, 2.5, 3, 3.5, 4, 4.5, 5, 6, 7, 8, 9, 10, 15, or 20 wt % or any range therein. In preferred aspects, the anti-hemorrhage ingredient can be aluminum sulfate, and the anti-acne agent can be salicylic acid or benzoyl peroxide. Further, other ingredients in a solid formulation may include additional active ingredients, including but not limited to anesthetics, depigmenting agents, analgesics, antihistamines, anti-inflammatory agents, and various botanical products. The compositions of the invention may include excipients. Excipients may include thickeners that enable formation of compositions that are thick enough that they hold their shapes but are able to deposit an effective amount of the composition when rubbed on skin. Such thickeners can include, as non-limiting examples, carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich). Non-limiting examples of crosslinked polyacrylate polymers include cationic and nonionic polymers. Examples are described in U.S. Pat. Nos. 5,100,660; 4,849,484; 4,835,206; 4,628,078; 4,599,379). Non-limiting examples of polyacrylamide polymers (including nomomc polyacrylamide polymers including substituted branched or unbranched polymers) include polyacrylamide, isoparaffin and laureth-7, multi-block copolymers of acrylamides and substituted acrylamides with acrylic acids and substituted acrylic acids. Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Non-limiting examples of gums that can be used with the present invention include acacia, agar, algin, alginic acid, ammonium alginate, amylopectin, calcium alginate, calcium carrageenan, camitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

The compositions of the present invention can also be formulated to be liquid, such as would be used in the applicator device illustrated in FIG. 2 or in FIG. 3. The active ingredients can be present in liquid formulations in any concentration effective to stop bleeding and promote healing of an acne lesion. Determining such effective concentrations is within the skill of those of ordinary skill in the art. The anti-hemorrhagic active ingredient may be included in amounts of 1, 2, 3, 4, 5, 6, 7, 8, 9, 10, 20, 30, 40, 50, 60, 70, or 80 wt % or any range therein. The anti-acne active ingredient may be included in the formulation in amounts of 0.5, 1, 1.5, 2, 2.5, 3, 3.5, 4, 4.5, 5, 6, 7, 8, 9, 10, 15, or 20 wt % or any range therein. In preferred aspects, the anti-hemorrhage ingredient can be aluminum sulfate, and the anti-acne agent can be salicylic acid or benzoyl peroxide. A liquid formulation should have a relatively low viscosity (e.g., less than 1,000,000 cps, or more preferably less than 100,000, or even more preferably less than 50,000 cps, 40,000 cps, 30,000 cps, 20,000 cps, 10,000 cps, 5,000 cps, or less than 1,000 cps, as measured on a Brookfield Viscometer using a TC spindle at 2.5 rpm at 25° C. to allow for flow of the composition from the reservoir through the wick and end piece to the skin. Other ingredients in a liquid formulation may include additional active ingredients, including but not limited to anesthetics, depigmenting agents, analgesics, antihistamines, anti-inflammatory agents, and various botanical products. Such a formulation can include such excipients which provide for compositions that are suited for delivery to skin via capillary action.

### C. Methods of Use

The methods of the present invention provide a way to use the applicator devices of the present invention to deliver an effective amount of a composition with both anti-hemorrhagic and anti-acne active ingredients to stop bleeding and promote healing of open acne lesions. The composition is delivered to an open acne lesion by contacting the lesion with the applicator device. In embodiments wherein the active ingredient composition is formed into an applicator stick, such as the embodiment shown in FIG. 1, a portion of the applicator stick 11 itself is deposited on the open lesion. In embodiments with a liquid active ingredient composition, as in the embodiment shown in FIG. 2, the composition is delivered to the open lesion via capillary action from the reservoir 22 through the wick 21 and applicator end piece 20 when the applicator end piece is placed on the subject's skin. For the embodiment in FIG. 3, the applicator tip 32 is simply dipped into a liquid formulation, absorbed by the tip 32, and then applied to the lesion.

The material may be left on the lesion for a treatment period, during which the active ingredients act on the open lesion. The treatment period may last for any period of time sufficient to allow the active ingredients to stop bleeding and promote healing of the lesion. At the end of the treatment period the active ingredient composition can be washed off with any suitable liquid including, for example, water or soapy water.

In the above description, numerous specific details are provided to provide a thorough understanding of the disclosed embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Other objects, features and advantages of the present invention will become apparent from the above detailed description. It should be understood, however, that the detailed description, while indicating specific embodiments of the invention, are given by way of illustration only. Additionally, it is contemplated that changes and modifications within the spirit and scope of the invention will become apparent to those skilled in the art from this detailed description.

The claims are not to be interpreted as including means-plus- or step-plus-function limitations, unless such a limitation is explicitly recited in a given claim using the phrase(s) “means for” or “step for,” respectively.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

The Table 1 formulation can be in the form of a paste.

TABLE 1*

| Ingredient | % (w/w) |
|---|---|
| Aluminum sulfate | 70 |
| Water | 20 |
| Anti-acne agent | 10 |
| Total | 100 |

*The composition can be prepared by combining aluminum sulfate with water, followed by mixing to form a paste. The anti-acne agent can be added to the paste with mixing. Increasing the water to aluminum sulfate ratio can decrease the viscosity of the formulation such that it is more liquid than paste-like. Decreasing the water to aluminum sulfate ratio can increase the viscosity of the formulation such that it is more solid than paste-like. In addition, other ingredients (active and inactive) can be added by simply modifying the water amount or aluminum sulfate amount or the amount of the anti-acne agent in the formulation. Additional excipients can also be added to help solubilize anti-acne agents that may be poorly soluble in water (e.g., salicylic acid, benzoyl peroxide, tretinoin, etc.). Such excipients include urea and sodium citrate, which can be used alone or in combination with other cosolvents (e.g., glycerin, propylene glycol, PEG 300 and PEG 400). The excipients can be added as needed to obtain the desired level of solubility of the anti-acne agent and the amount of water in the formulation can be adjusted accordingly.

The Table 2 formulation can be in the form of a liquid.

TABLE 2*

| Ingredient | % (w/w) |
|---|---|
| Aluminum sulfate | 20 |
| Water | 70 |
| Anti-acne agent | 10 |
| Total | 100 |

*The composition can be prepared by combining aluminum sulfate with water, followed by mixing to form a paste. The anti-acne agent can be added to the paste with mixing. Increasing the water to aluminum sulfate ratio can decrease the viscosity of the formulation such that it is more liquid than paste-like. Decreasing the water to aluminum sulfate ratio can increase the viscosity of the formulation such that it is more solid than paste-like. In addition, other ingredients (active and inactive) can be added by simply modifying the water amount or aluminum sulfate amount or the amount of the anti-acne agent in the formulation. Additional excipients can also be added to help solubilize anti-acne agents that may be poorly soluble in water (e.g., salicylic acid, benzoyl peroxide, tretinoin, etc.). Such excipients include urea and sodium citrate, which can be used alone or in combination with other cosolvents (e.g., glycerin, propylene glycol, PEG 300 and PEG 400). The excipients can be added as needed to obtain the desired level of solubility of the anti-acne agent and the amount of water in the formulation can be adjusted accordingly.

The Table 3 formulation can be in the form of a solid.

TABLE 3*

| Ingredient | % (w/w) |
|---|---|
| Aluminum sulfate | 90 |
| Water | 7 |
| Anti-acne agent | 3 |
| Total | 100 |

*The composition can be prepared by combining aluminum sulfate with water, followed by mixing to form a paste. The anti-acne agent can be added to the paste with mixing. Increasing the water to aluminum sulfate ratio can decrease the viscosity of the formulation such that it is more liquid than paste-like. Decreasing the water to aluminum sulfate ratio can increase the viscosity of the formulation such that it is more solid than paste-like. In addition, other ingredients (active and inactive) can be added by simply modifying the water amount or aluminum sulfate amount or the amount of the anti-acne agent in the formulation. Additional excipients can also be added to help solubilize anti-acne agents that may be poorly soluble in water (e.g., salicylic acid, benzoyl peroxide, tretinoin, etc.). Such excipients include urea and sodium citrate, which can be used alone or in combination with other cosolvents (e.g., glycerin, propylene glycol, PEG 300 and PEG 400). The excipients can be added as needed to obtain the desired level of solubility of the anti-acne agent and the amount of water in the formulation can be adjusted accordingly.

Tables 4-6 include liquid styptic formulations from U.S. Pat. No. 4,166,108, that have been modified by including 2% salicylic acid as the anti-acne agent and reducing the water amount by a corresponding 2%. Otherwise, the formulations can be prepared by mixing the ingredients in a container to produce the resulting formulation. Additional actives (e.g., skin lightening or brightening ingredients) can be added to these formulations by decreasing the corresponding amount of water in said formulations.

TABLE 4

| Ingredient | % (w/w) |
|---|---|
| Stearic acid | 7.5 |
| Beeswax | 1.7 |
| Polyethylene glycol stearate | 5.3 |
| Polyethylene glycol sorbitan beeswax | 3.5 |
| Aluminum sulfate | 13 |
| Salicylic acid | 2 |
| Water | 67 |

TABLE 5

| Ingredient | % (w/w) |
|---|---|
| Palmitic acid | 9 |
| Microcrystalline wax | 2.5 |
| Polyethylene glycol stearate | 3.8 |
| Polyethylene glycol sorbitan beeswax | 2.3 |
| Aluminum sulfate | 10 |
| Salicylic acid | 2 |
| Water | 70.4 |

TABLE 6

| Ingredient | % (w/w) |
|---|---|
| Lauric acid | 11 |
| Paraffin wax | 3 |
| Polyethylene glycol stearate | 6.2 |
| Polyethylene glycol sorbitan beeswax | 4 |
| Zinc sulfate | 18 |
| Salicylic acid | 2 |
| Water | 55.8 |

All of the devices, skin-active ingredients, compositions, or methods disclosed and claimed in this specification can be made and executed without undue experimentation in light of the present disclosure. While the skin-active ingredients, compositions, or methods of this invention have been described in terms of particular embodiments, it will be apparent to those of skill in the art that variations may be applied to the skin-active ingredients, compositions, or methods and in the steps or in the sequence of steps of the method described herein without departing from the concept, spirit and scope of the invention.

### REFERENCES

The following references, to the extent that they provide exemplary procedural or other details supplementary to those set forth herein, are specifically incorporated herein by reference.
U.S. Pat. No. 459,738
U.S. Pat. No. 2,798,053
U.S. Pat. No. 5,087,445
U.S. Pat. No. 4,509,949
U.S. Pat. No. 4,599,379
U.S. Pat. No. 4,628,078
U.S. Pat. No. 4,835,206
U.S. Pat. No. 4,849,484
U.S. Pat. No. 5,100,660

1. A styptic composition formulated for topical application to an acne lesion, the composition comprising an anti-acne agent and a sufficient amount of a styptic to seal the lesion within 30 seconds after topical application of the composition to the acne lesion.

2. The styptic composition of claim 1, comprising 50 to 95% by weight of the styptic and 1 to 10% by weight of the anti-acne agent.

3. The styptic composition of claim 2, further comprising 10 to 50% by weight of water.

4. The styptic composition of claim 1, comprising 10 to 50% by weight of the styptic and 1 to 10% by weight of the anti-acne agent.

5. The styptic composition of claim 4, further comprising 50 to 90% by weight of water.

6. The styptic composition of claim 1, wherein the composition is formulated as a solid stick.

7. The styptic composition of claim 1, wherein the composition is formulated as a semi-solid.

8. The styptic composition of claim 7, wherein the composition has a viscosity of greater than 150,000 cps as measured by Brookfield Viscometer using a TC spindle at 2.5 rpm at 25° C.

9. The styptic composition of claim 1, wherein the composition is formulated as a liquid.

10. The styptic composition of claim 9, wherein the composition has a viscosity of less than 150,000 cps as measured by Brookfield Viscometer using a TC spindle at 2.5 rpm at 25° C.

11. The styptic composition of claim 1, wherein the styptic is aluminum sulfate and the anti-acne agent is salicylic acid or benzoyl peroxide.

12. The styptic composition of claim 1, wherein the styptic is aluminum sulfate, potassium alum, titanium dioxide, or zinc chloride, or any combination thereof.

13. The styptic composition of claim 12, wherein the anti-acne agent is an antibacterial agent (e.g., azelaic acid, benzoyl peroxide, or hydroxyquinoline, or combinations thereof), a keratolytic agent (e.g., glycolic acid, salicylic acid, benzoyl peroxide, or combinations thereof), an antibiotic (e.g., clindamycin, dapsone, erythromycin, sulfacetamide, tetracyclines such as lymecycline, minocycline, doxycycline, or combinations thereof), a retinoid (e.g., adapalene, isotretinoin, motretinide, tazarotene, or tretinoin, or combinations thereof), mesulfen, or tioxolone, or any combination thereof.

14. The styptic composition of claim 1, further comprising a skin lightening ingredient.

15. The styptic composition of claim 1, further comprised in a device configured to apply the composition to the acne lesion.

16. The styptic composition of claim 15, wherein the composition is in solid form and is disposed lengthwise within an elongated housing of said device.

17. The styptic composition of claim 15, wherein the composition is in liquid form and comprised within a reservoir of the device, wherein the device further comprises an absorbent applicator end piece in fluid communication with said reservoir.

18. A method of sealing or treating an acne lesion on a person's skin comprising contacting the acne lesion with a composition of claim 1 to said acne lesion, wherein said composition seals the acne lesion within 30 seconds after application.

19. The method of claim 18, wherein the acne lesion is bleeding when contacted with the composition and stops bleeding within 30 second after contact.

20. The method of claim 18, wherein the composition is applied to the acne lesion via an absorbent applicator end piece of an applicator device.
